(19) United States
(12) Patent Application Publication
(10) Pub. No.: US 2018/0132990 A1
(43) Pub. Date: May 17, 2018

(54) AUTOMATED CORDLESS SYRINGE

(71) Applicant: Inter-Med, Inc., Racine, WI (US)

(72) Inventors: John BAETEN, Oak Creek, WI (US); Brett A. ARAND, Hubertus, WI (US); Gary J. POND, Milwaukee, WI (US)

(21) Appl. No.: 15/567,015

(22) PCT Filed: Apr. 14, 2016
(86) PCT No.: PCT/US2016/027435

§ 371 Date: Oct. 16, 2017

Related U.S. Application Data
(60) Provisional application No. 62/147,061, filed on Apr. 14, 2015.

(51) Int. Cl. A61C 17/34; A61C 5/40; A61M 5/32; A61M 5/142; A61M 3/02; A61C 17/02

(52) U.S. Cl. CPC A61C 17/34; A61C 5/40; A61M 5/3202; A61M 5/14228; A61M 3/0258; A61C 17/0217; A61M 2205/6072; A61M 2205/0294; A61M 2205/3334; A61M 2205/8206; A61M 2205/36; A61M 2205/3337; A61M 5/14212

(57) ABSTRACT

A device for delivering fluid includes a housing having a proximal end configured to receive a fluid source and a distal end configured to receive a treatment tip. In its interior, the housing includes at least a first fluid line in fluid communication with the fluid source and with the treatment tip, at least a first pump in-line with the first fluid line and configured to urge fluid from the fluid source to the treatment tip, control circuitry in electrical communication with the pump, and a power source configured to supply continuous output power to deliver the fluid to the treatment tip, the power source being in electrical communication with the control circuitry. The housing further is provided with user-operable controls and operation monitors.

This application claims the benefit of priority from U.S. provisional application 62/147,061, filed Apr. 14, 2015, the disclosure of which is incorporated herein by reference in its entirety.

### BACKGROUND OF THE INVENTION

In medical, surgical, dental, and other fields (e.g., endodontics, periodontics, personal hygiene), fluid is delivered to intended treatment sites using standard manual syringes at desired flow rates. While such delivery can be simple and effective, undesired effects, including hand fatigue, inconsistent flow, and unsafe high positive pressure at a treatment site can occur. Also, reliance upon disposable standard manual syringes generates waste, requires more storage space, and increases cost. Furthermore, syringes either need to be purchased prefilled, or an operator manually fills the syringes with medicaments before use, adding time to procedures and generating waste. There also are increased safety risks with the traditional syringe, including the introduction of air bubbles into delivery and unregulated flow rate.

Powered syringes in a handheld device can deliver small, metered doses of a medicament. Powered desktop pumping units can infuse fluids into a recipient over an extended time. Neither type of powered device exists in a small, simple to use, and convenient package with a wide range of chemical compatibility, and neither type of device can deliver variable fluid volumes over short time periods. Both also typically require a traditional syringe to be attached instead of self priming and drawing fluid from a reservoir.

### SUMMARY OF THE INVENTION

The present invention provides an economical, ergonomic, lightweight, compact, cordless, convenient, easy-to-use, automated device for dispensing and delivering a multitude of fluids at a constant, consistent flow rate to a treatment site in, e.g., medical, surgical, and dental applications without the need for a traditional syringe. For dental applications, suitably deliverable fluids include, without limitation, sodium hypochlorite, ethylenediaminetetraacetic acid (EDTA), chlorhexidine based solutions, oral rinses, saline, alcohols, air and other common dental fluids. For medical applications, the device may incorporate a medicinal cartridge for injection, pump insulin or other medications, or precision delivery of medicament, aggregates, adhesives, or other substrates used during diagnostic, treatment, or reconstructive procedures.

The device generally includes, in a housing configured to be comfortably and stably held by the operator, at least one internal fluid line that connects a quick-connect fluid source and a treatment tip or needle that meets the various dimensional constraints and flow rate requirements of distinct treatment sites. At least one fluid pump in fluid connection with the at least one internal fluid line is dimensionally, directionally, and positionally configured to directly or indirectly urge fluid from the fluid source to the treatment tip. The fluid source is designed in such a way that air bubbles are controlled and that fluid can be drawn in any spatial orientation of the device. The at least one pump is electronically controlled, is powered by an on-board power source, and meets the size, power, pressure, and flow rate requirements of the device and the application. The targeted flow rate for this device is at least 4 mL/min irrespective of the gauge of the needle, but this may range from 0.5 mL/min to 10 mL/min for endodontic procedures or medicinal delivery, or it may be increased above 10 mL/min, up to 100 mL/min, for periodontal cleaning procedures. The power source provides power output sufficiently high to ensure that the at least one fluid pump operates at suitable drive parameters (including but not limited to voltage, duty cycle, frequency, current, rise and fall time, and other defining waveform characteristics) to provide for the desired fluid flow rate. User-actuated control circuitry is in electronic connection with the fluid pump(s). User-actuated switches or other structures for activating and deactivating the device, for adjusting drive parameters to the pump (and, thus, the flow rate), and for monitoring operational parameters can be provided on the device housing, remote from the housing, or in other ways known in the art.

### BRIEF DESCRIPTION OF THE DRAWINGS

FIG. 1 is a top view of the hand held fluid delivery device with an attached fluid source according to the present invention.

FIG. 2 is a cut-away view an embodiment of the hand held fluid delivery device shown in FIG. 1 that employs a single pump.

FIG. 3 is a cut-away view of another embodiment of the hand held fluid delivery device shown in FIG. 1 that employs two pumps in series.

FIG. 4 is a cut-away view of yet another embodiment of the hand held fluid delivery device shown in FIG. 1 that employs two pumps in parallel.

FIG. 5 is a cut-away view of still another embodiment of the hand held fluid delivery device shown in FIG. 1 that employs a pump to pressurize the fluid reservoir with air.

FIG. 6 is a cut-away view of another embodiment of the hand held fluid delivery device shown in FIG. 1 that employs a single pump and an external fitting to connect a vacuum line for fluid evacuation.

FIG. 7 is a cut-away view of a further embodiment of the hand held fluid delivery device shown in FIG. 1 that employs one pump for fluid delivery and one pump for fluid evacuation.

FIG. 8A is top view of one embodiment of a treatment tip that performs irrigation and evacuation through the same orifice.

FIG. 8B is a top view of one embodiment of a treatment tip that performs irrigation and evacuation through separate orifices.

FIG. 9A is one example of a fluid source usable with at least one of the embodiments described above.

FIG. 9B is a second example of a fluid source usable with at least one of the embodiments described above.

FIG. 9C is a third example of a fluid source usable with at least one of the embodiments described above.

FIG. 9D is a fourth example of a fluid source usable with at least one of the embodiments described above.

FIG. 10 depicts an experimental setup for determining flow rates and back pressures encountered using various treatment tips.

FIG. 11 depicts a general circuit diagram for the device of FIG. 1.

FIG. 12 is a graph depicting back pressure versus flow rate for needles having various gauges.

FIG. 13 is a graph depicting flow rate versus needle gauge size for various pump configurations.

### DETAILED DESCRIPTION OF THE INVENTION

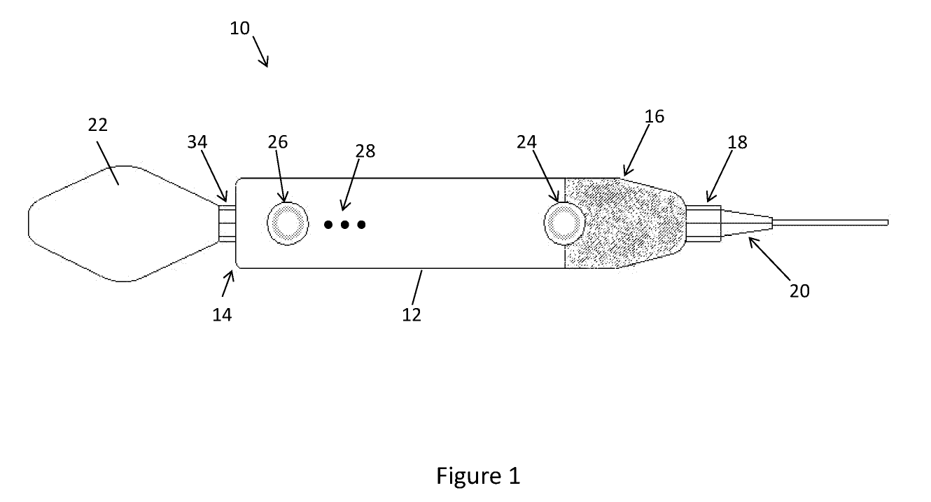

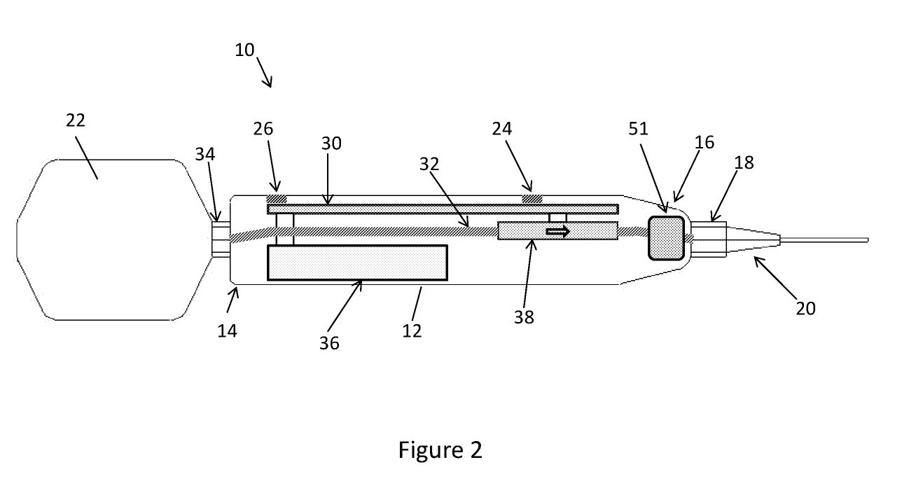

FIG. 1 and FIG. 2 illustrate an embodiment of a cordless fluid delivery device 10 according to the present invention. The device 10 generally comprises a rigid housing 12 having a proximal end 14 configured to be held by the user and a distal end 16. A fitting 18, such as a Luer lock or Luer slip fitting, is provided at the distal end 16 to receive a treatment tip 20, also referred to as a needle. The tip can be a conventional straight or pre-bent tip. The housing 12 can optionally be provided with a protective cover that can be, without limitation, a flexible silicone sleeve, a hard plastic member, or a plastic or metal extension tube, on at least a portion of its exterior surface. If provided, the protective cover can be discarded after a single use or can be removed for sterilization and repositioned at the distal end of the housing 12. The housing 12 is provided with a proximal end fitting 34 that can receive an on-board or remote fluid source 22. On/off control 24, a flow rate selector 26, and a flow rate indicator 28 are also provided on the exterior of the housing 12.

As seen in FIG. 2, provided within the housing 12 is control circuitry 30, in electrical or mechanical-electrical communication with the on/off control 24, the selector 26, and the indicator 28, that directs and controls power to the device 10. Some or all of the control circuitry 30 can be provided, e.g., on an internal printed circuit board. The circuitry generally includes a power source 36, a voltage transformer, a voltage inverter, user inputs, and at least one protective component, such as a pressure transducer to shut off flow in the event of an occlusion or a sensor to only allow the use of approved fluids. A sensor and feedback loop can monitor flow rate and modify the drive waveform accordingly to achieve a desired flow rate or to overcome back pressure created by an attached tip 20.

The power source 36 can include at least one single use (primary) or rechargeable battery or super capacitor 36 capable of supplying continuous output power of at least 0.2 W during operation for at least about 20 minutes of use. The battery can be removable and replaceable. The battery can be lithium ion, nickel cadmium, alkaline or the like and is preferably a single cell rechargeable lithium ion battery. A rechargeable battery or super capacitor can be rechargeable by direct electrical contact charging or by induction charging.

The fluid source 22, here a reservoir preferably having a sealable fluid outlet connector, is in direct or indirect fluid communication with the fittings 18 and 34, and with the treatment tip 20 via one or more internal fluid lines 32. The fluid source 22 can connect to the fluid line 32 via a fluid source connector 34, and can, as needed, be detached, reattached, replaced, and/or interchanged with another fluid source. The fluid source connector 34 can be, without limitation, a threaded, Luer lock, quick-connect, or similar fitting known to a skilled artisan. The fluid source 22, fluid line 32, tips 20, fittings, and any other fluid-contacting element should be chemically compatible with the fluid(s).

At least one fluid pump 38, also in electrical communication with the control circuitry 30, is interposed in the one or more fluid lines 32. The at least one pump 38 is advantageously self-priming, compact, and quiet, and has low operational power consumption requirements, typically between about 0.1 W and 2 W, preferably less than 500 mW. Desired run time to be an effective product is at least 30 minutes but up to 2 hours from a single charge of the rechargeable battery. For a 500 mW power consumption, this would require a minimum 250 mAh battery for 30 minutes of use and up to a 1 Ah battery for 2 hours of use. If a single pump provides inadequate flow rate or fails to overcome back pressure encountered at the treatment tip 20, a plurality of pumps 38 can be provided, as discussed in greater detail below and as shown in the figures. The at least one pump can be a piezoelectric pump or an electric (DC) motor pump such as a peristaltic or diaphragm pump.

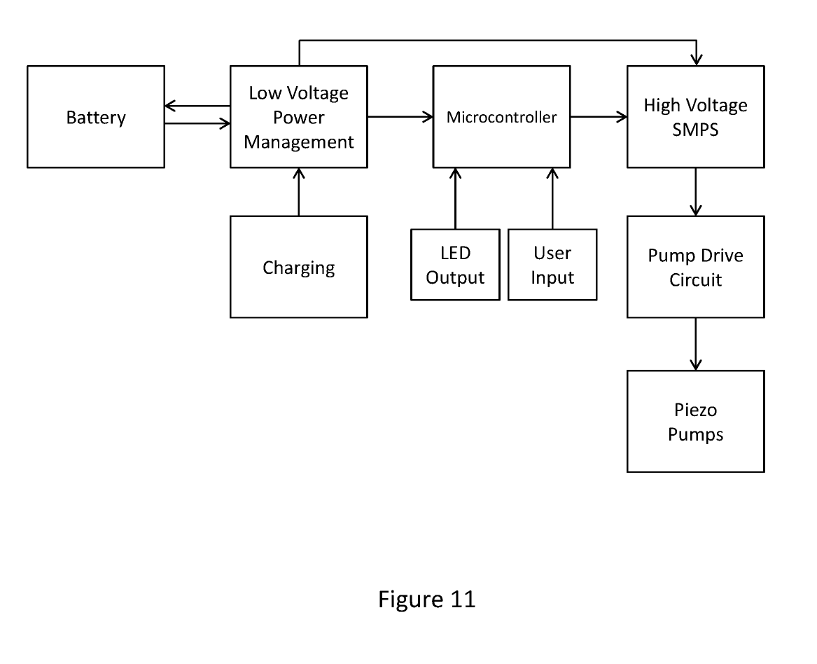

Piezoelectric pumps operate using a piezoelectric diaphragm in combination with passive check valves. A piezo ceramic mounted on a membrane is deformed when voltage is applied. The deformation causes displacement of the medium, liquid or gas, within the pump chamber. The check valves cause the medium to always be drawn in from one end, and expelled from the other, so that flow is created in the desired direction. Some of the advantages to a piezoelectric pump include low power consumption, small size, reliability of priming and prevention of occluding, and availability at an economically viable price, although they may be limited by flow and pressure capabilities. The piezo crystal responds to an AC voltage with the maximum and minimum of the waveform causing the crystal to displace in both “up” and “down” direction from its steady state position. The drive may be specific to the pump manufacturer, though a typical drive would be 100-400 Volts peak-to-peak waveform operating at 20-1000 Hz. The type of electrical waveform may be, but is not limited to, sinusoidal, square, or exponential ramp, and may include a DC offset. The drive waveform is based on requirements of the pump selected and a balance between performance, audible noise, and pump longevity. The peak-to-peak voltage has a positive correlation with both flow rate and pressure handling of the pump. Depending on the manufacturers design, inappropriate voltages in the positive or negative may depolarize the piezo crystals, resulting in reduced performance. Too high of a voltage also may run the risk of electrical breakthroughs through the ceramic and jumping between electrodes. A slower rise time on the waveform will make the pumps audibly quieter, whereas a faster rise time may increase the performance of the pump; however, there is a limit as too sharp of a rise time can lead to cracking of the piezo crystal. Waveform frequency may increase the flow rate of the pump; however, the maximum frequency is limited by the recovery time of the check valves. Flow rate can be selectable by the user by adjusting at least one of duty cycle, frequency, waveform shape, and waveform peak-to-peak voltage if using piezoelectric pumps. A general circuit diagram for the device utilizing one or more piezoelectric pumps is shown in FIG. 11. Suitable piezoelectric pumps for this device are manufactured by, e.g., Takasago Fluidic Systems, Bartels Mikrotechnik, Murata and MicroJet Technology Co.

Electric (DC) motor pumps designed for fluid delivery are typically of a diaphragm or peristaltic type. A DC diaphragm pump uses the reciprocating action of a diaphragm membrane in combination with check valves to cause positive displacement of the medium. The diaphragm flexes and creates negative pressure to draw in the medium, then flexes in the other direction to cause the medium to displace. Diaphragm displacement can be done with a DC motor and piston or other linear drive methods. The check valves ensure that the medium is only drawn in through one end and expelled from the other. Advantages of DC diaphragm pumps may include availability in a wide range of pressure and flow ratings and utility for a variety of fluids, including liquids and gas; however, if DC-driven, they may not be as compact or low power as the piezoelectric type of pump. A peristaltic pump uses a rotor with one or more rollers that compresses flexible tubing as it rotates inside a circular pump casing. As the tubing is pinched, it is closed off and the roller is able to push the medium forward through the line. The corresponding negative pressure created behind the roller then draws in the new medium from the inlet. Advantages to peristaltic pumps are low risk of occlusion, ability to handle highly viscous materials, and a high level of compatibility with different mediums since only the tubing is wetted. However, the pumps are typically larger and require more power, and the tubing used will wear out over time from squeezing and may need to be replaced. An electric (DC) motor pump would be driven by DC voltage ranging from 0 Volts to the maximum voltage recommended for the chosen pump with a duty cycle of 0-100%. Flow rate can be selectable by the user by adjusting at least one of DC voltage or duty cycle if using an electric (DC) motor pump. Suitable electric (DC) motor pumps are manufactured by, e.g., Parker, KNF, Schwarzer Precision, TCS Micropumps.

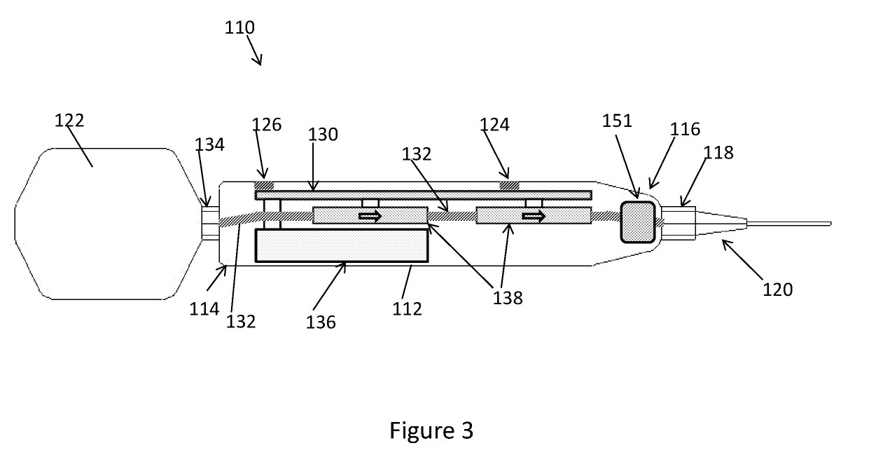

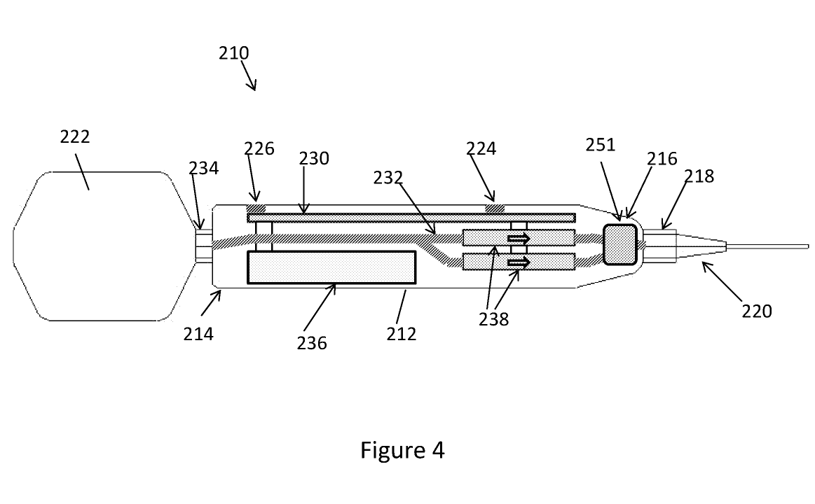

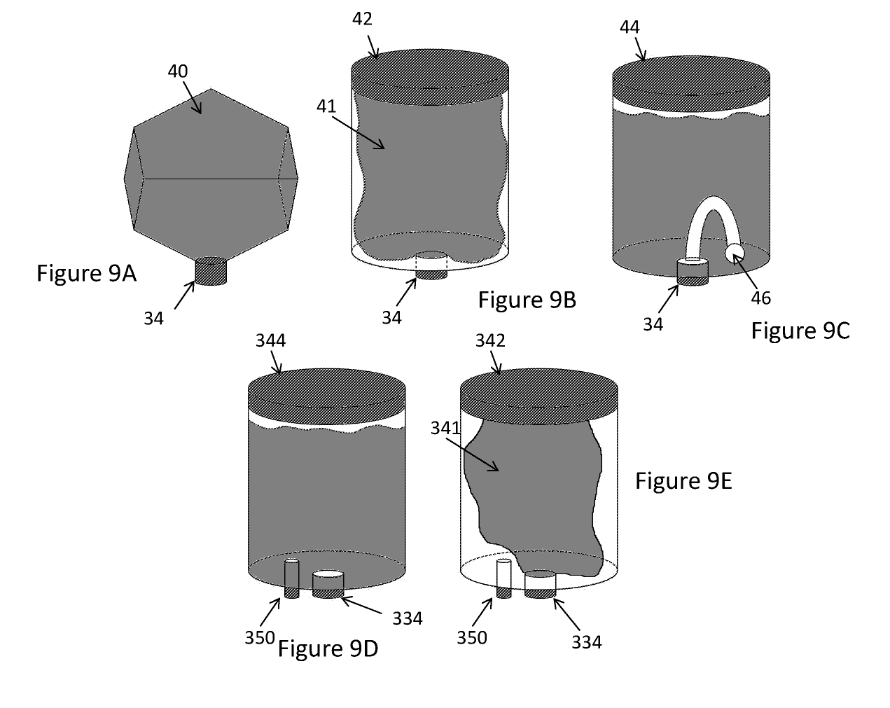

Various embodiments employing other combinations of pumps and fluid line arrangements are envisioned, including the ones depicted in the figures. In those embodiments, like reference numerals to the embodiment of FIGS. 1 and 2, albeit with different prefixes, represent like elements. FIG. 3 illustrates a related embodiment of the device 110 in which two pumps 138 are provided in a single fluid line 132 in series to increase the total pumping pressure. FIG. 4 illustrates a second related embodiment of the device 210 in which two pumps 238 are provided in parallel in separate fluid line branches 232 to increase the total flow rate. In these configurations, the pump(s) 138, 238 can self-prime the empty fluid line 132, 232 and draw fluid into the line by creating a negative pressure environment in the fluid source 122, 222, which can be, for example, a flexible pouch 40 (see FIG. 9A) that collapses as the fluid drains, a hard canister 42 having a collapsible internal pouch 41 (FIG. 9B), or a hard canister 44 provided with an internal drinking tube 46 in fluid communication with the outlet of the fluid source 22 (FIG. 9C). All fluid reservoir embodiments allow fluid to be drawn from the pouch in any orientation of the device while preventing or limiting the intake of air bubbles into the fluid line. Each fluid line 32 can be provided with one or a plurality of pumps 38. In all embodiments described above and below, a heater block 51, 151, etc., can be implemented to heat the fluid in-line 32, 132, etc., before it is delivered out the tip 20, 120, etc., as fluid heating may have significant benefits on the efficacy of the pumped fluid. (Sirtes, et al., “The Effects of Temperature on Sodium Hypochlorite Short-Term Stability, Pulp Dissolution Capacity, and Antimicrobial Efficacy,” Journal of Endodontics 31.9: 669-671 (2005)). Additionally, the fluid source can be provided prefilled to help prevent introduction of air bubbles into the line, which is disadvantageous to pump performance and may be harmful to the patient depending on the intended use of the device.

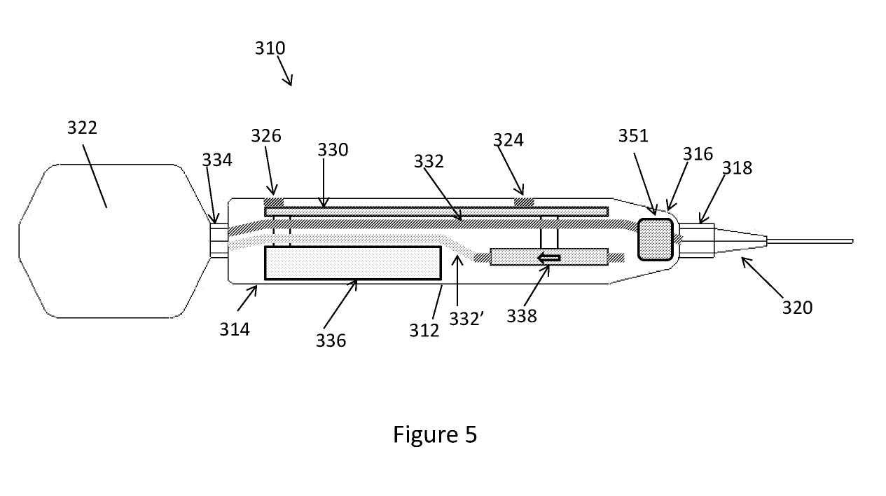

Alternatively, as shown in FIG. 5, a fluid line 332′ provided with at least one in-line pump 338 can be configured to force a sufficient volume of gaseous fluids through the fluid line 332′ into the fluid source containing fluid (344, in FIG. 9D) or containing a flexible fluid-containing pouch 341 (342, as in FIG. 9E) to increase pressure on the liquid in the fluid source 322 thereby forcing liquid from the fluid source 322 though a separate fluid line 332 toward and into the treatment tip 320. In such a configuration, the fluid source 322 would be provided with an air inlet 350, which would receive gas pumped through fluid line 332′ into the fluid source 322 (either into the liquid directly through, for example a one-way check valve, or, as in FIG. 9E, into a space surrounding a flexible container 341 holding liquid), thereby pressurizing the liquid and, in either case, forcing liquid from the fluid source 322 through fluid line 332 toward and into the treatment tip 320. In this embodiment, the fitting (not shown) could connect the fluid source 322 to both fluid lines 332 and 332′. In any configuration of the fluid source 322, it is envisioned that the fluid source 322 may have a quick connect fitting to mate with the device 310 to quickly replace when empty or switch between types of fluids. When removed from the device 310, the fluid source 322 may feature a valve to prevent fluid from leaking out or air from entering. The valve may also allow for refilling of the fluid source by attaching to another device, such as a traditional syringe or a custom designed filling station such as the filling station disclosed in the commonly assigned Pond et al., U.S. Pat. No. 8,671,994 and U.S. Publication No. 2010/0059140, the disclosures of which both are incorporated herein by reference. Also, the fluid source 322 may be designed in such a way that the device is able to draw fluid from any orientation without the introduction of air to the line.

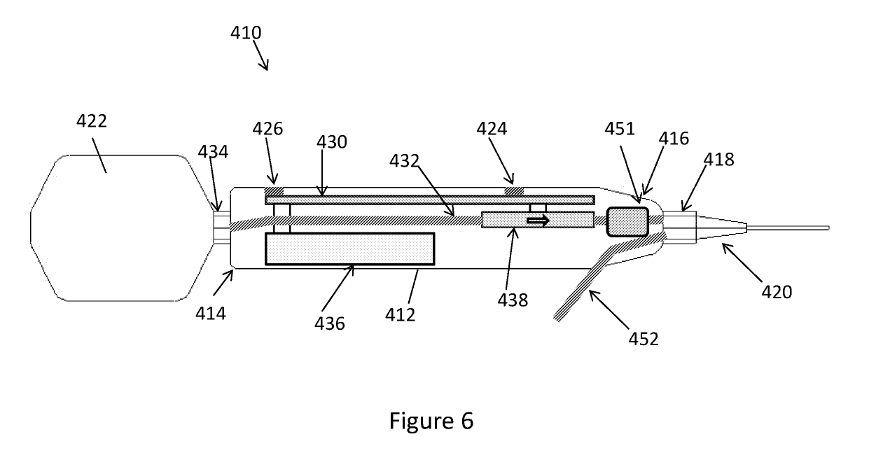

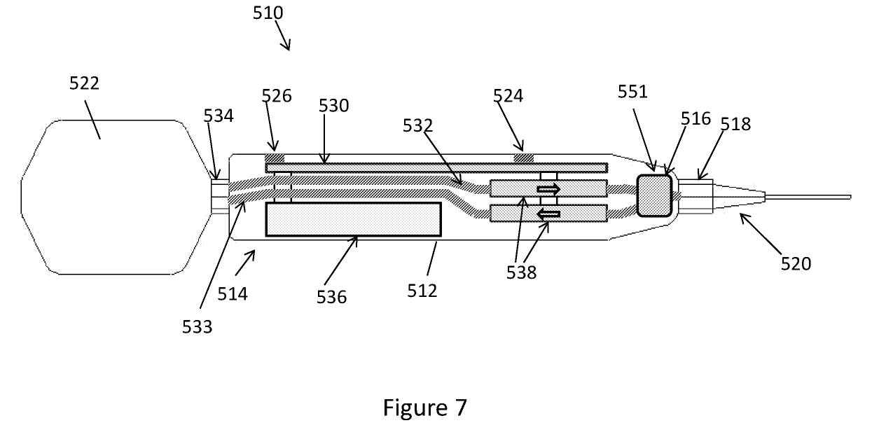

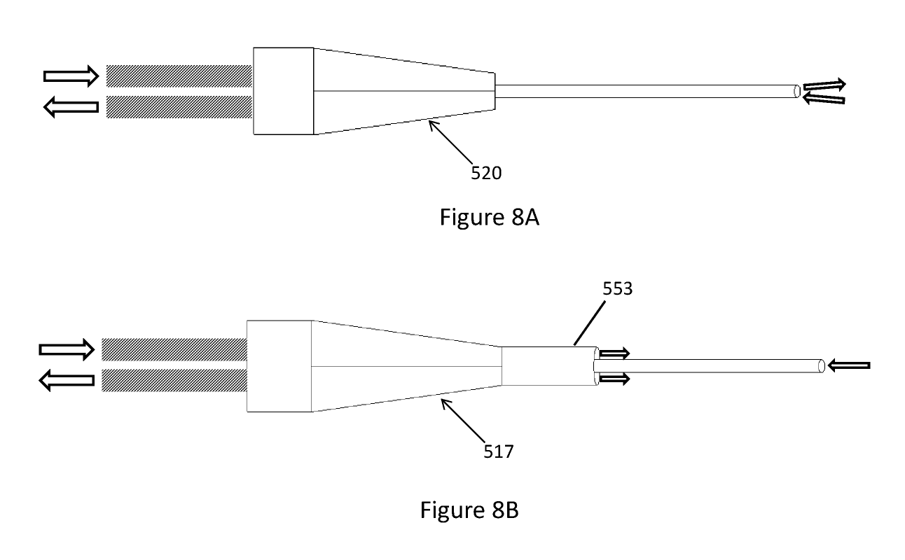

In still other embodiments, the device also may incorporate a connection for an external vacuum line 452 (see FIG. 6), a two pump system 538 (see FIG. 7) or a single pump system 38 (see FIG. 2) that is able to reverse its flow direction for fluid evacuation. In the case of the two pump system 538, the evacuation line 533 could fill an external fluid reservoir 22 or drain through a tube via an external connection. In one aspect, as seen in FIG. 8A, evacuation could be done using a traditional needle tip 520 through the same fluid path and tip 520 that irrigation is performed, e.g., using valves to control flow direction in each type of use. In this case, the device may be configured to only perform irrigation or evacuation at one time. In another aspect, as seen in FIG. 8B, an evacuation pathway may be completely separate from the irrigation pathway, which may be accomplished through the use of a specialized front sleeve 517 and tip 553 that incorporate the two separate fluid paths instead of a single pathway. The fluid pathways may be registered together in a way that the two cannot be mixed up, thereby avoiding cross-contamination with each other. Irrigation and evacuation could be designed to occur at the same length on the tip 553 or at different lengths. Fluid evacuation can be used for either general removal of fluid from the working space or for negative pressure irrigation, wherein fluid is irrigated from a higher point in working space and evacuated at the bottom of the workspace (e.g., near the canal apex in a dental application) (Khan, et al., “Periapical Pressures Developed by Nonbinding Irrigation Needles at Various Irrigation Delivery Rates,” Journal of Endodontics 39.4: 529-533 (2013)).

The fluid source and device may also interact with each other in a way that the programming inside the device receives information about the fluid that is attached. The information can be coded on the canister in multiple ways. Simple color coding or grayscale can be used to identify the solution, e.g., a light may reflect off a chosen color paint, sticker, dye, or other colorant and be picked up by a photodiode. Based on how much light that color reflects, software executed by processor on the device is able to determine type of solution. Alternatively, a bar or QR code or similar may be scanned with a laser reader or optical camera, the bar or QR code or similar having a large amount of information encoded, including but not limited to the type of solution, expiration date, and an identifier unique to that specific canister to monitor if it has been used before. In still another alternative, a passive RF tag or other form of near field communication can be installed on the canister and sensed by the device when the canister is installed. As with the bar or QR coding, this option also may allow for a large amount of information to be stored. Still further, some fluids may become ineffective after the expiration date, and the device can be programmed to prevent the use of such solutions. It also may be unsafe to use certain fluids in sequence, since they may mix and have an undesired chemical reaction with each other. Thus, the device may be configured to monitor the type and order of fluids used and analyze that information against data corresponding to prohibited or undesirable combinations in order to prevent such mixing. Also, only approved fluids may be used with the device, and one or more of the techniques described herein may be used to determine that the container is from an acceptable source and/or to track if the container has been used before to prevent refills, thereby preventing the use of untested fluids that may be harmful to the device or patient or otherwise may be outside of the application scope of the device.

The electronic control circuitry 30 provides the operator with variable or selectable control of fluid flow rate. To ensure that the desired flow rate is achieved, control circuitry 30 can monitor the flow rate via a feedback loop and can adjust the flow rate by varying the fluid pump drive parameters. By way of example, flow rates appropriate and safe for use in endodontic cleansing range from about 0.5 mL/min to 15 mL/min, and more preferably from about 0.5 mL/min to about 10 mL/min. For endodontic work, about 4 mL/min is a preferred flow rate of solutions delivered into a root canal by positive pressure irrigation syringe via a general use irrigation needle (Park, “Apical pressure and extent of irrigant flow beyond the needle tip during positive-pressure irrigation in an in vitro root canal model,” Journal of Endodontics 39.4: 511-515 (2013)), as there is no greater clearance of solutions at higher rates. Also, at higher flow rates the apical pressure generated during endodontic cleansing can cause irrigation solutions to penetrate beyond the apex into body tissue (Park 2013). Hypochlorite accident, i.e., expression of sodium hypochlorite (NaOCl) beyond the confines of the root canal, can result in significant patient harm. In another example, flow rates higher than 10 mL/min may have utility in periodontal application that don't have the risk of harm from positive pressure.

### EXAMPLES

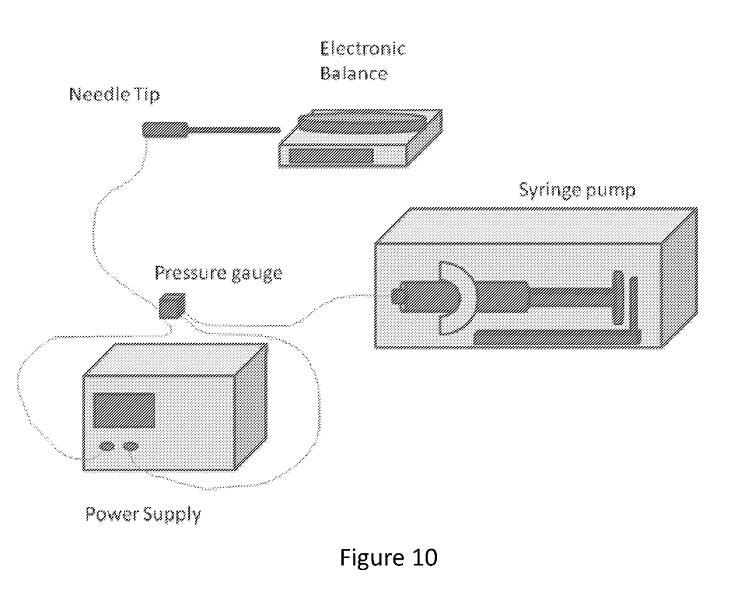

We determined suitable fluid pump flow-rates for a device in accord with the invention by measuring the back pressures created in 23, 27, and 30 gauge needles when liquid was pumped at various defined flow rates (0-15 mL/min) from a 60 ml syringe using an infusion syringe pump (All Pro Corporation, Model WZ-50C6T, modified to remove occlusion limiter). A pressure gauge (Honeywell True Stability Model SSCDANN030PGAA5) in direct communication with the fluid line recorded the pressures that developed at various flow rates. The experimental setup is depicted in FIG. 10.

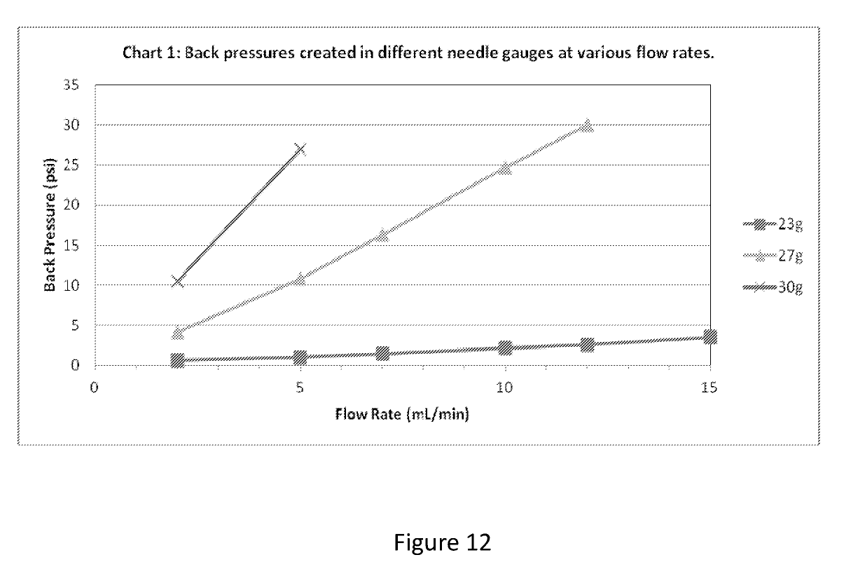

Results are shown in Chart 1 (FIG. 12). We observed a positive correlation between back pressure and flow rate, which was expected from the following equation for change in pressure in a tube, which shows a direct relationship between pressure and flow rate.

$$Δ P = \frac{128 μ LQ}{π {a}^{4}}$$

where:

ΔP—is the change in pressure

L—is the length of pipe

μ—is the dynamic viscosity

Q—is the volumetric flow rate

r—is the radius

d—is the inner diameter

π—is the mathematical constant Pi

Small gauge needles can achieve suitably high flow rates only if the pumping system can overcome high back pressure. Flow rate and back pressure created are linearly related. One can determine from the linear slope the required back pressure tolerance of a pump at a desired flow rate through a given tip. These results show that suitable fluid pumps must handle back pressures exceeding 10 psi and upwards of 30 psi to deliver suitable flow rates through commonly used needle tips. Flow rates could not be analyzed for some needle gauges because the infusion pump could not create pressures above 35 pounds per square inch (psi). With a 23 gauge needle, the back pressure remained under 5 psi when tested up to 15 mL/min. The 27 gauge needle could only be tested to 12 mL/min, at which rate it produced 30 psi of pressure. The 30 gauge needle caused much greater pressure, reaching 27 psi at only 5 mL/min. This can also be explained by the above equation, as the inner diameter is raised to the 4^{th} power. The listed flow rates are for liquids; suitable flow rates for gases would range up to 20 L/min.

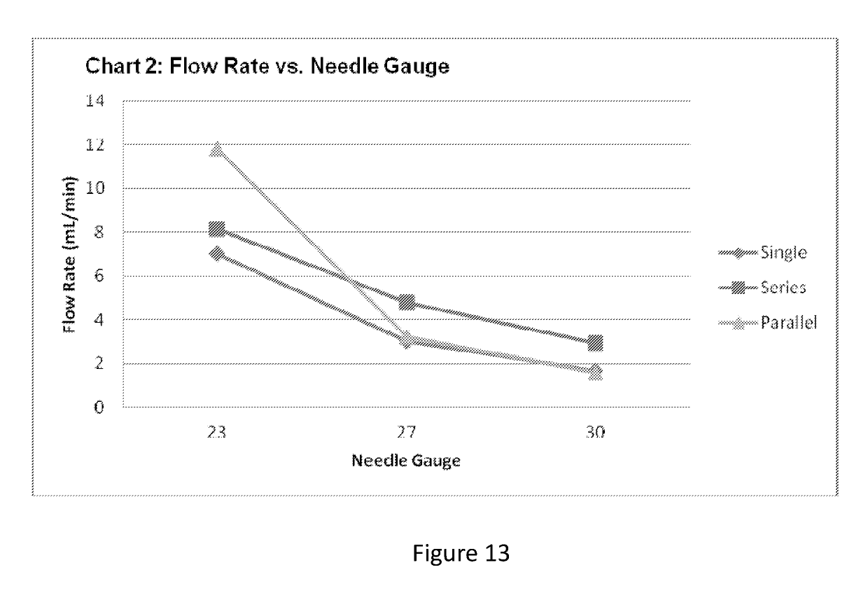

To determine configurations appropriate to achieve suitable flow rates using one or more piezoelectric pump (Bartels Mikrotechnik mp6), flow rate was measured for each of the three needle gauges in various configurations, including a single pump, two pumps with fluid paths in series, and two pumps with fluid paths in parallel, as shown above. The tubing used was 1/16 inch ID Tygon PVC, with 180 mm length at the inlet and outlet. For the parallel configuration, 10 mm of tubing was provided between the pumps and the Y-connector. For the series configuration, 10 mm of tubing was provided between the two pumps. Chart 2 (FIG. 13) shows that two Bartels Mikrotechnik mp6 piezoelectric pumps connected in series can provide desired flow rates from commonly used needle gauges.

The foregoing description was primarily directed to one or more embodiments of the invention. Although some attention has been given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. A device for delivering fluid, the device comprising:
a housing having a proximal end configured to receive a fluid source and a distal end configured to receive a treatment tip,
the housing comprising in its interior:
at least a first fluid line in fluid communication with the fluid source and with the treatment tip;
at least a first piezoelectric pump in-line with the first fluid line and configured to urge fluid from the fluid source to the treatment tip;
control circuitry responsive to user input and configured to direct an electrical waveform to the pump to balance pump performance, audible pump noise, and pump longevity, the waveform being adjustable to vary pump fluid flow output rate; and
a power source configured to supply continuous output power to deliver the fluid to the treatment tip, the power source being in electrical communication with the control circuitry.

2. The device of claim 1, wherein the housing further comprises in its interior:
a second pump in-line with the first fluid line and configured to urge fluid from the fluid source to the treatment tip.

3. The device of claim 1, wherein the housing further comprises in its interior:
a second fluid line in fluid communication with the fluid source and with the treatment tip; and
a second pump in-line with the second fluid line and configured to urge fluid from the fluid source to the treatment tip.

4. The device of claim 1, wherein a plurality of fluid lines is provided with a plurality of in-line pumps.

5. The device of claim 1, further comprising at least one of an on/off switch and a flow rate selector.

6. The device of claim 1, further comprising a flow rate indicator.

7. The device of claim 1, further comprising a a protective cover on at least a portion of the device.

8. The device of claim 7, wherein the protective cover is selected from the group consisting of a flexible silicone sleeve, a hard plastic member, and a plastic or metal extension tube.

9. The device of claim 7, wherein the protective cover can be sterilized.

10. The device of claim 7, wherein the protective cover is disposable.

11. (canceled)

12. The device of claim 1, wherein the power source is selected from a single use (primary) battery and a rechargeable battery.

13. The device of claim 1, wherein the power source is selected from the group consisting of a lithium ion battery, a nickel cadmium battery, an alkaline battery, and a super capacitor.

14. The device of claim 1, wherein the device comprises a plurality of fluid lines for delivering fluid to the treatment tip, each fluid line being provided with an in-line pump configured to urge fluid from the fluid source to the treatment tip.

15. The device of claim 1, wherein the device further comprises a fluid line for delivering a gas to the fluid source and a pump in-line with the fluid line, the pump configured to urge gas to the fluid source.

16. The device of claim 15, wherein the fluid line for delivering the gas terminates in fluid present in the fluid source.

17. The device of claim 15, wherein the fluid line for delivering the gas terminates in a space surrounding a fluid container in the fluid source.

18. The device of claim 1, wherein the device further comprises a heater block disposed in-line between the at least first pump and the treatment tip.

19. The device of claim 1, wherein the device further comprises a fluid line configured for evacuation.

20. The device of claim 19, wherein the at least one fluid line and the fluid line configured for evacuation are configured to avoid cross-contamination with one another.

21. The device of claim 1, wherein the device is cordless.

22. The device of claim 1, wherein the fluid source is removable.

23. The device of claim 1, wherein the control circuitry comprises a high voltage switch mode power supply.

24. The device of claim 23, wherein the power supply is in direct electrical communication with the power source.

25. The device of claim 1, wherein the waveform has an adjustable frequency for directing the variable fluid flow output rate.
